(19) United States
(12) Patent Application Publication
(10) Pub. No.: US 2026/0015353 A1
(43) Pub. Date: Jan. 15, 2026

(54) NOVEL COMPOUNDS

(71) Applicant: Hoffmann-La Roche Inc., Little Falls, NJ (US)

(72) Inventors: Lea Aurelie BOUCHE, Basel (CH); Wolfgang GUBA, Muellheim (DE); Georg JAESCHKE, Basel (CH); Stefanie Katharina MESCH, Basel (CH); Angélique PATINY-ADAM, Kembs (FR); Christian SCHNIDER, Biel-Benken (CH); Sandra STEINER, Sursee (CH); Andreas Michael TOSSTORFF, Muenchenstein (CH)

(73) Assignee: Hoffmann-La Roche Inc., Little Falls, NJ (US)

(21) Appl. No.: 18/996,517

(22) PCT Filed: Jul. 19, 2023
(86) PCT No.: PCT/EP2023/069977

§ 371 Date: Jan. 17, 2025

(30) Foreign Application Priority Data
Jul. 21, 2022 (EP) 22186134.7

(51) Int. Cl. C07D 471/04; A61K 31/53

(52) U.S. Cl. CPC C07D 471/04; A61K 31/53

(57) ABSTRACT

The invention relates to novel compounds having the general formula (I), wherein R¹, R², R³, R⁴ and W are as described herein, composition including the compounds and methods of using the compounds.

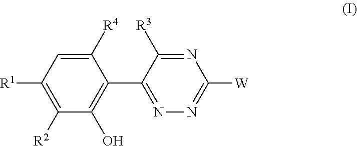

### FIELD OF THE INVENTION

The present invention relates to organic compounds useful for therapy and/or prophylaxis in a mammal, and in particular to compounds that modulate NLRP3 inhibition.

The present invention provides novel compounds of formula I

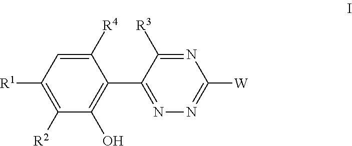

wherein
R¹ is H, halo, alkyl, haloalkyl, haloalkoxy or cyano;
R² is H;
or R¹ and R², and the atoms to which they are bonded, form a 4-6 membered cycloalkyl or a 4-6 membered heterocycle ring, wherein the cycloalkyl and the heterocycle ring are optionally substituted with one or two substituents independently selected from halo, alkyl or oxo;
R³ is selected from H, alkyl, cyano or haloalkyl, and R⁴ is selected from H, alkyl, halo, cyano or —CH₂-alkoxy, wherein R³ and R⁴ are not both H;
W is an 8-10 membered N-linked bicyclic ring system or an 8-10 membered N-linked spirocyclic ring system, wherein the N-Linked bicyclic ring system or N-Linked spirocyclic ring system comprise at least 2 N heteroatoms and can be optionally substituted by up to four substituents independently selected from hydroxyl, alkyl, halogen or cyano;
and pharmaceutically acceptable salts thereof.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R² resulting in its degradation and allowing the release of IL-la. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

### SUMMARY OF THE INVENTION

The present invention provides novel compounds of formula I:

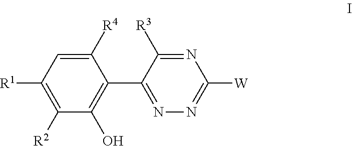

wherein
R¹ is H, halo, alkyl, haloalkyl, haloalkoxy or cyano;
R² is H;
or R¹ and R², and the atoms to which they are bonded, form a 4-6 membered cycloalkyl or a 4-6 membered heterocycle ring, wherein the cycloalkyl and the heterocycle ring are optionally substituted with one or two substituents independently selected from halo, alkyl or oxo;
R³ is selected from H, alkyl, cyano or haloalkyl, and R⁴ is selected from H, alkyl, halo, cyano or —CH₂-alkoxy, wherein R³ and R⁴ are not both H;
W is an 8-10 membered “N-linked” bicyclic ring system or an 8-10 membered “N-linked” spirocyclic ring system, wherein the N-Linked bicyclic ring system or N-Linked spirocyclic ring system comprise at least 2 N heteroatoms and can be optionally substituted by up to four substituents independently selected from hydroxyl, alkyl, halogen or cyano;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “alkoxy” denotes a group of the formula-O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy.

The term “cyano” denotes a —C═N group.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. In other embodiments, cycloalkyl comprises one or more double bonds (e.g., cycloalkyl fused to an aryl or heteroaryl ring, or a non-aromatic monocyclic hydrocarbon comprising one or two double bonds). Examples of cycloalkyl groups include cyclopropyl, cyclobutyl, cyclopentyl, cyclohexyl, octahydropentalenyl, spiro[3.3]heptanyl, and the like. Examples for monocyclic cycloalkyl are cyclopropyl, cyclobutanyl, cyclopentyl, cyclohexyl or cycloheptyl.

The term “halogen”, “halide” and “halo” are used interchangeably herein and denote fluoro, chloro, bromo or iodo.

The term “haloalkyl” denotes a C₁₋₆-alkyl group wherein at least one of the hydrogen atoms of the C₁₋₆-alkyl group has been replaced by the same or different halogen atoms.

The term “haloalkoxy” denotes a C₁₋₆-alkoxy group wherein at least one of the hydrogen atoms of the C₁₋₆-alkoxy group has been replaced by the same or different halogen atoms.

The term “heterocycle” denotes a monovalent saturated or partly unsaturated mono- or bicyclic ring system of 4 to 10 ring atoms, or 4 to 9 ring atoms, comprising 1, 2, or 3 ring heteroatoms selected from N, O and S, the remaining ring atoms being carbon. Examples for monocyclic saturated heterocycle rings are oxetanyl, azetidinyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, or piperazinyl. Examples for partly unsaturated heterocycle rings are dihydrofuryl, imidazolinyl, dihydro-oxazolyl, tetrahydro-pyridinyl, or dihydropyranyl. Particular example of a heterocycle ring is tetrahydrofuran.

The term “N-Linked bicyclic ring system” denotes a heterocycle system up to 10 ring atoms comprising two rings having two carbon atoms in common, wherein the bicyclic ring system attached to the rest of the molecule through an N-heteroatom. Examples of an N-Linked bicyclic ring system include (6-ethyl)-3,3a,4,5,7,7a-hexahydro-2H-pyrrolo[2,3-c]pyridine-1-yl.

The term “N-Linked spirocyclic ring system” denotes a heterocycle system of up to 10 ring atoms comprising two rings which are conjoined at a single carbon atom, and wherein the spirocyclic ring system is attached to the rest of the molecule through an N-heteroatom.

The term “hydroxyl” denotes an —OH group.

The term “oxo” denotes a refers to a doubly bonded oxygen (—O), i.e. a carbonyl group.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is cyano and R² is H, or R¹ and R², and the atoms to which they are bonded, form a 4-6 membered heterocycle ring comprising a single O heteroatom optionally substituted with one or two substituents independently selected from halo or alkyl.

An embodiment of the present invention provides compounds according to formula I as described herein, wherein R¹ is cyano and R² is H, or R¹ and R², and the atoms to which they are bonded, form a 5 membered heterocycle ring comprising a single O heteroatom.

An embodiment of the present invention provides compounds according to formula I as described herein, wherein R³ is H or alkyl.

An embodiment of the present invention provides compounds according to formula I as described herein, wherein R³ is alkyl.

An embodiment of the present invention provides compounds according to formula I as described herein, wherein R⁴ is H.

An embodiment of the present invention provides compounds according to formula I as described herein, wherein W is selected from ring systems A, B, or C

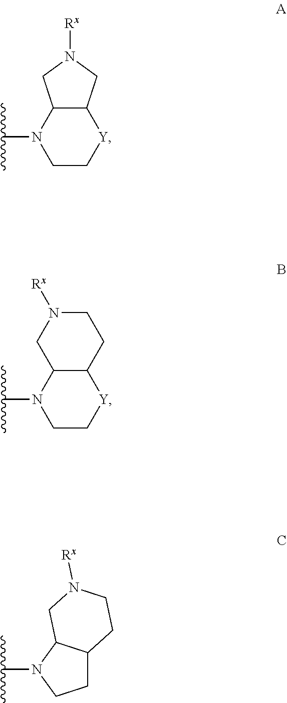

wherein
Y is CH₂, O, or NR^{y};
R^{y} is H or alkyl;
R^{x} is H or alkyl;
and the ring system A, B, or C can be further substituted with one cyano or up to two halo substituents.

An embodiment of the present invention provides compounds according to formula I as described herein, wherein W is selected from ring systems A, B, or C

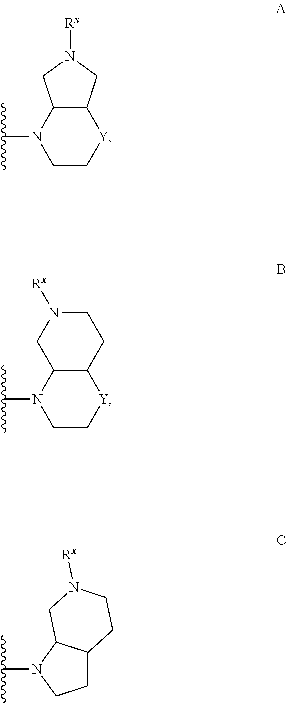

wherein Y is CH₂, or O and R^{x} is H or alkyl.

An embodiment of the present invention provides compounds according to formula I as described herein, wherein W is ring system C

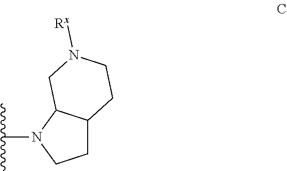

wherein R^{x} is alkyl.

An embodiment of the present invention provides compounds according to formula I, wherein
R¹ is cyano and R² is H, or R¹ and R², and the atoms to which they are bonded, form a 5 membered heterocycle ring comprising a single O heteroatom;
R³ is alkyl;
R⁴ is H;
W is ring system C

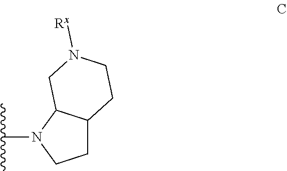

R^{x} is alkyl;
and pharmaceutically acceptable salts thereof.

Particular examples of compounds of formula I as described herein are selected from
5-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
5-[3-[(3aR,7aS)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aS,7aR)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
4-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile;
4-[3-[(3aR,7aS)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aS,7aR)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile;
5-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
5-[3-[(3aR,7aS)-6-Methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aS,7aR)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
4-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-3-hydroxy-benzonitrile;
4-[3-[(3aR,7aS)-6-Methyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aS,7aR)-6-methyl-3,3a,4,5,7,7a-hexahydro-2H-pyrrolo[2,3-c]pyridin-1-yl]-5-methyl-1,2,4-triazin-6-yl]-3-hydroxy-benzonitrile;
   and pharmaceutically acceptable salts thereof.

Preferred examples of compounds of formula I as described herein are selected from
5-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
5-[3-[(3aR,7aS)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aS,7aR)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
4-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile;
5-[3-[(3aS,7aR)-6-Methyl-3,3a, 4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
   and pharmaceutically acceptable salts thereof.

Processes for the manufacture of compounds of formula I as described herein are an object of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166:1-15, 2011; Strowig et al., Nature, 481:278-286, 2012).

In one embodiment, the disease, disorder or condition is selected from:
(i) inflammation;
(ii) an auto-immune disease;
(iii) cancer,
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder,
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer,
(ii) an infection;
(iii) a central nervous system disease;
(iv) a cardiovascular disease;
(v) a liver disease;
(vi) an ocular disease; or
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;
(vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is the use of a compound according to formula I as described herein in the treatment or prophylaxis of a disease, disorder or condition selected from Alzheimer's disease and Parkinson's disease.

An embodiment of the present invention is the use a compound according to formula I as described herein for use in the treatment or prophylaxis of a disease, disorder or condition selected from Asthma or COPD.

An embodiment of the present invention is the use a compound according to formula I as described herein for use in the treatment or prophylaxis of a disease, disorder or condition selected from inflammatory bowel disease (including Crohn's disease and ulcerative colitis).

An embodiment of the present invention is a compound according to formula I as described herein for the treatment or prophylaxis of a disease, disorder or condition selected from Alzheimer's disease and Parkinson's disease.

An embodiment of the present invention is a compound according to formula I as described herein for the treatment or prophylaxis of a disease, disorder or condition selected from Asthma or COPD.

An embodiment of the present invention is a compound according to formula I as described herein for the treatment or prophylaxis of a disease, disorder or condition selected from inflammatory bowel disease (including Crohn's disease and ulcerative colitis).

An embodiment of the present invention is the use of a compound according to formula I as described herein for preparation of a medicament for the treatment or prophylaxis of a disease, disorder or condition selected from Alzheimer's disease and Parkinson's disease.

An embodiment of the present invention is the use of a compound according to formula I as described herein for the preparation of a medicament for the treatment or prophylaxis of a disease, disorder or condition selected from Asthma or COPD.

An embodiment of the present invention is the use of a compound according to formula I as described herein for the preparation of a medicament for the treatment or prophylaxis of a disease, disorder or condition selected from inflammatory bowel disease (including Crohn's disease and ulcerative colitis).

An embodiment of the present invention is a method of treatment or prophylaxis of a disease, disorder or condition selected from Alzheimer's disease and Parkinson's disease, which method comprises administering an effective amount of a compound according to formula I as described herein.

An embodiment of the present invention is a method of treatment or prophylaxis of a disease, disorder or condition selected from Asthma or COPD, which method comprises administering an effective amount of a compound according to formula I as described herein.

An embodiment of the present invention is a method of treatment or prophylaxis of a disease, disorder or condition selected from inflammatory bowel disease (including Crohn's disease and ulcerative colitis), which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

An embodiment of the present invention is a compound according to formula I as described herein for the treatment or prophylaxis of a disease, disorder or condition selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8 (2), e2579; Alexander Wree et al., Hepatology, 2014, 59 (3), 898-910; Alex Baldwin et al., Journal of Medicinal Chemistry, 2016, 59 (5), 1691-1710; Ema Ozaki et al., Journal of Inflammation Research, 2015, 8, 15-27; Zhen Xie & Gang Zhao, Neuroimmunology Neuroinflammation, 2014, 1 (2), 60-65; Mattia Cocco et al., Journal of Medicinal Chemistry, 2014, 57 (24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000cells/ml. To this diluted cell solution was added LPS (Sigma #L4524) to give a lug/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hours at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R⁷⁰¹⁷) (FAC 100 μM resazurin in RPMI medium without FBS) and incubate plates for a further 1-2 hours at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing lug/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hours at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

TABLE 1
NLRP3 inhibitory activity

| | THP-1 pyroptosis assay | Human whole blood IL-1β |
|---|---|---|
| Example No. | IC₅₀ (nM) | Assay IC₅₀ (nM) |
| 1A | 1.8 | 12 |
| 1B | 64.4 | 375 |
| 2A | 36.4 | 38 |
| 2B | >1000 | — |
| 3A | 1.9 | 7 |
| 3B | 52.2 | 115 |
| 4A | 47.4 | 41 |
| 4B | >1000 | — |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen atmosphere if not specified otherwise.

### Abbreviations

| | |
|---|---|
| eq | equivalent(s) |
| ESI | Electrospray ionization |
| LCMS | Liquid chromatography mass spectrometry |
| SFC | Supercritical Fluid Chromatography |

### Example 1A and 1B

### 5-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol and 5-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol

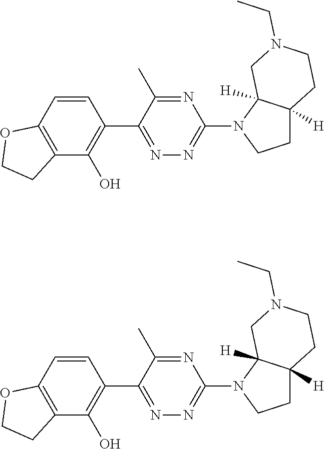 [SMILES: CCN1CC[C@H]2CCN(c3nnc(-c4ccc5c(c4O)CCO5)c(C)n3)[C@H]2C1 ; CCN1CC[C@@H]2CCN(c3nnc(-c4ccc5c(c4O)CCO5)c(C)n3)[C@@H]2C1]

### Step A: tert-Butyl 1-(6-chloro-5-methyl-1,2,4-triazin-3-yl)-3,3a,4,5,7,7a-hexahydro-2H-pyrrolo[2,3-c]pyridine-6-carboxylate

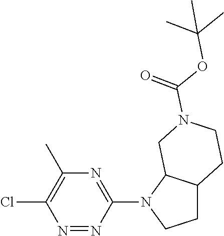 [SMILES: Cc1nc(N2CCC3CCN(C(=O)OC(C)(C)C)CC32)nnc1Cl]

To a mixture of 3,6-dichloro-5-methyl-1,2,4-triazine (CAS #132434-82-3, 500 mg, 3.05 mmol, 1.00 eq) and 1,2,3,3a,4,5,7,7a-octahydropyrrolo[2,3-c]pyridine-6-carboxylic acid tert-butyl ester (CAS #1196147-27-9, 898 mg, 3.97 mmol, 1.30 eq) in 1,4-dioxane (9.0 mL) was added N,N-diisopropylethylamine (414 mg, 0.56 mL, 3.21 mmol, 1.05 eq). The reaction mixture was stirred at room temperature for 16 hours. The reaction mixture extracted with ethyl acetate and water. The aqueous layer was backextracted with ethyl acetate. The organic layers were washed with brine. The combined organic layers were dried over sodium sulfate, filtered and concentrated in vacuo.

The crude product was purified by flash chromatography (silica gel, 40 g, gradient 0% to 30% ethyl acetate in heptane) to afford the title compound (952 mg, 84% yield) as a yellow oil. LCMS: m/z 354.2 [M+H]⁺, ESI pos.

### Step B: 1-(6-Chloro-5-methyl-1,2,4-triazin-3-yl)-2,3,3a,4,5,6,7,7a-octahydropyrrolo[2,3-c]pyridine hydrochloride

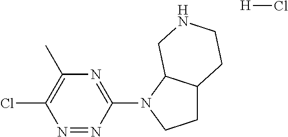

To a solution of tert-butyl 1-(6-chloro-5-methyl-1,2,4-triazin-3-yl)-3,3a,4,5,7,7a-hexahydro-2H-pyrrolo[2,3-c]pyridine-6-carboxylate (Example 1, step A) (938 mg, 2.52 mmol, 1.00 eq) in dichloromethane (8.0 mL) and methanol (4.0 mL) was added dropwise 4 M HCl in dioxane (6 mL, 24 mmol, 9.53 eq). The reaction mixture was stirred at room temperature for 2 hours. The reaction mixture was concentrated in vacuo to afford the title compound (1.09 g, 97% yield, 65% purity) as an orange foam, which was used without further purification. LCMS: m/z 254.2 [M+H]⁺, ESI pos.

### Step C: 1-(6-Chloro-5-methyl-1,2,4-triazin-3-yl)-6-ethyl-3,3a,4,5,7,7a-hexahydro-2H-pyrrolo[2,3-c]pyridine

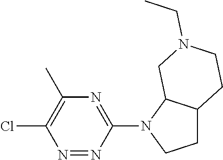 [SMILES: CCN1CCC2CCN(c3nnc(Cl)c(C)n3)C2C1]

To a suspension of 1-(6-chloro-5-methyl-1,2,4-triazin-3-yl)-2,3,3a,4,5,6,7,7a-octahydropyrrolo[2,3-c]pyridine hydrochloride (Example 1, step B) (500 mg, 1.12 mmol, 1.00 eq, 65% purity) in dichloromethane (7.0 mL) was added acetaldehyde (125 mg, 0.160 mL, 2.83 mmol, 2.53 eq) followed by sodium acetate (185 mg, 2.26 mmol, 2.01 eq) under ice-bath cooling. Sodium triacetoxyborohydride (358 mg, 1.69 mmol, 1.51 eq) was added in three portions at 0° C. After the addition was complete, the ice-bath was removed and the reaction mixture was stirred at room temperature for 1 hour. The reaction mixture was carefully quenched with saturated aq. NaHCO₃-solution and extracted three times with dichloromethane. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane) to afford the title compound (246 mg, 74% yield) as a yellow oil. LCMS: m/z 282.2 [M+H]⁺, ESI pos.

### Step D: 4-Benzyloxy-5-bromo-2,3-dihydrobenzofuran

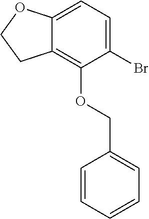 [SMILES: Brc1ccc2c(c1OCc1ccccc1)CCO2]

To a solution of 5-bromocoumaran-4-ol (CAS #2279149-27-6, 4.59 g, 20.3 mmol, 1.00 eq) in acetonitrile (40 mL) was added potassium carbonate (5.60 g, 40.5 mmol, 2.00 eq) followed by benzyl bromide (4.89 g, 3.4 mL, 28.6 mmol, 1.41 eq). The reaction mixture was stirred at room temperature for 2 hours. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 220 g, gradient 0% to 10% ethyl acetate in heptane) to afford the title compound (6.17 g, 95% yield) as a colorless oil. LCMS: m/z 305.1/307.0 [M+H]⁺, ESI pos.

### Step E: 2-(4-benzyloxy-2,3-dihydrobenzofuran-5-yl)-4,4,5,5-tetramethyl-1,3,2-dioxaborolane

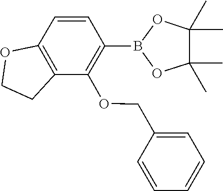 [SMILES: CC1(C)OB(c2ccc3c(c2OCc2ccccc2)CCO3)OC1(C)C]

To a solution of 4-benzyloxy-5-bromo-2,3-dihydrobenzofuran (Example 1, step D) (6.16 g, 19.2 mmol, 1.00 eq) and 2-isopropoxy-4,4,5,5-tetramethyl-1,3,2-dioxaborolane (CAS #61676-62-8, 5.47 g, 6.0 mL, 29.41 mmol, 1.53 eq) in tetrahydrofuran (80 mL) was added dropwise n-butyllithium, 1.6 M solution in hexanes (19 mL, 30.4 mmol, 1.59 eq) within 40 minutes at −76° C. Let stir at −76° C. for 2.5 hours. The reaction mixture was warmed to −60° C., quenched with saturated aq. NH₄Cl-solution at −60° C., warmed to room temperature and then extracted with ethyl acetate and saturated aq. NH₄Cl-solution. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 120 g, gradient 0% to 10% ethyl acetate in heptane) to afford the title compound (5.78 g, 81% yield) as a colorless oil. LCMS: m/z 353.1 [M+H]⁺, ESI pos.

### Step F: 5-(4,4,5,5-Tetramethyl-1,3,2-dioxaborolan-2-yl)-2,3-dihydrobenzofuran-4-ol

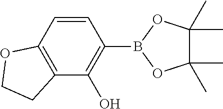 [SMILES: CC1(C)OB(c2ccc3c(c2O)CCO3)OC1(C)C]

A solution of 2-(4-benzoxycoumaran-5-yl)-4,4,5,5-tetramethyl-1,3,2-dioxaborolane (5.77 g, 15.56 mmol, 1.00 eq) in ethyl acetate (70 mL) was three times alternating evacuated and flushed with argon. Palladium on activated charcoal, 10% Pd basis (577 mg, 0.54 mmol, 0.03 eq) was added carefully. The reaction flask was evacuated, flushed with argon, evacuated and flushed with hydrogen. The reaction mixture was stirred under hydrogen atmosphere (balloon) at room temperature for 3 hours. Methanol (10 mL) was added. The reaction flask was three times alternating evacuated and flushed with argon, evacuated and then flushed with hydrogen. The reaction mixture was stirred under hydrogen atmosphere (balloon) at room temperature for 1 hour. The reaction mixture was filtered and rinsed well with ethyl acetate/methanol. The filtrate was concentrated in vacuo to afford the title compound (4.22 g, 98% yield) as an off-white solid, which was used without further purification. LCMS: m/z 263.2 [M+H]⁺, ESI pos.

### Step G: 5-[3-(6-Ethyl-3,3a,4,5,7,7a-hexahydro-2H-pyrrolo[2,3-c]pyridin-1-yl)-5-methyl-1,2,4-triazin-6-yl]-2,3-dihydrobenzofuran-4-ol

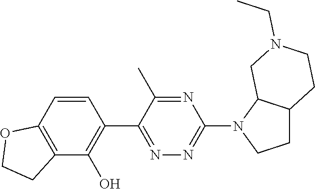 [SMILES: CCN1CCC2CCN(c3nnc(-c4ccc5c(c4O)CCO5)c(C)n3)C2C1]

A mixture of 1-(6-chloro-5-methyl-1,2,4-triazin-3-yl)-6-ethyl-3,3a,4,5,7,7a-hexahydro-2H-pyrrolo[2,3-c]pyridine (Example 1, step ( ) (120 mg, 0.40 mmol, 1.00 eq), 5-(4,4,5,5-tetramethyl-1,3,2-dioxaborolan-2-yl)-2,3-dihydrobenzofuran-4-ol (Example 1, step F) (179 mg, 0.65 mmol, 1.60 eq), potassium carbonate (257 mg, 1.86 mmol, 4.60 eq) and 1,1′-bis(diphenylphosphino) ferrocene-palladium(II) dichloride dichloromethane complex (49 mg, 0.06 mmol, 0.15 eq) in 1,4-dioxane (2.6 mL) and water (1.3 mL) was flushed with argon and stirred at 95° C. for 16 hours. The reaction mixture was cooled to room temperature and then extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (151 mg, 88% yield, 90% purity) as a dark brown foam. LCMS: m/z 382.2 [M+H]⁺, ESI pos.

### Step H: 5-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol and 5-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol

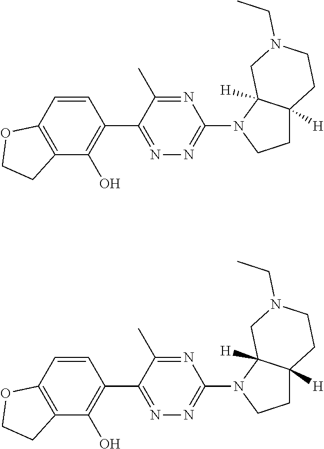 [SMILES: CCN1CC[C@H]2CCN(c3nnc(-c4ccc5c(c4O)CCO5)c(C)n3)[C@H]2C1 ; CCN1CC[C@@H]2CCN(c3nnc(-c4ccc5c(c4O)CCO5)c(C)n3)[C@@H]2C1]

Chiral separation by SFC (column chiral IK, eluent B: 36% methanol+0.2% diethylamine) of 5-[3-(6-ethyl-3,3a,4,5,7,7a-hexahydro-2H-pyrrolo[2,3-c]pyridin-1-yl)-5-methyl-1,2,4-triazin-6-yl]-2,3-dihydrobenzofuran-4-ol (Example 1, step G) (145 mg, 0.34 mmol, 1.00 eq) to afford the two enantiomers example 1A (first eluting, Rt=2.92 minutes) (63 mg, 46% yield) as a yellow foam; LCMS: m/z 382.3 [M+H]⁺, ESI pos and example 1B (second eluting, Rt=3.21 minutes) (53 mg, 39% yield) as a yellow foam; LCMS: m/z 382.3 [M+H]⁺, ESI pos.

### Example 2A and 2B

### 4-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile and 4-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile

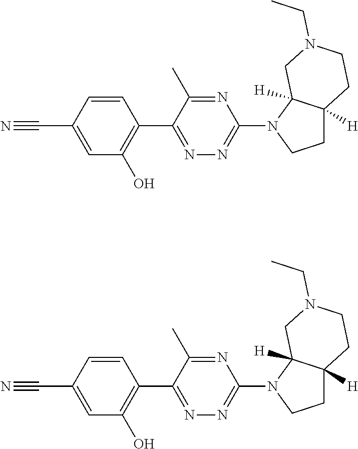 [SMILES: CCN1CC[C@H]2CCN(c3nnc(-c4ccc(C#N)cc4O)c(C)n3)[C@H]2C1 ; CCN1CC[C@@H]2CCN(c3nnc(-c4ccc(C#N)cc4O)c(C)n3)[C@@H]2C1]

### Step A: 4-[3-(6-Ethyl-3,3a,4,5,7,7a-hexahydro-2H-pyrrolo[2,3-c]pyridin-1-yl)-5-methyl-1,2,4-triazin-6-yl]-3-hydroxy-benzonitrile

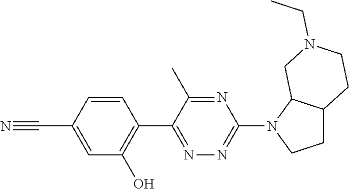 [SMILES: CCN1CCC2CCN(c3nnc(-c4ccc(C#N)cc4O)c(C)n3)C2C1]

A mixture of 1-(6-chloro-5-methyl-1,2,4-triazin-3-yl)-6-ethyl-3,3a,4,5,7,7a-hexahydro-2H-pyrrolo[2,3-c]pyridine (Example 1, step C) (99 mg, 0.33 mmol, 1.00 eq), (4-cyano-2-hydroxy-phenyl) boronic acid (commercially available, no CAS, 88 mg, 0.54 mmol, 1.62 eq), potassium carbonate (212 mg, 1.53 mmol, 4.60 eq) and 1,1′-bis(diphenylphosphino) ferrocene-palladium(II) dichloride dichloromethane complex (41 mg, 0.05 mmol, 0.15 eq) in 1,4-dioxane (2.0 mL) and water (1.0 mL) was flushed with argon and stirred at 90° C. overnight. The reaction mixture was cooled to room temperature and then extracted with ethyl acetate and half-saturated NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (62 mg, 48% yield) as a yellow foam. LCMS: m/z 365.2 [M+H]⁺, ESI pos.

### Step B: 4-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile and 4-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile

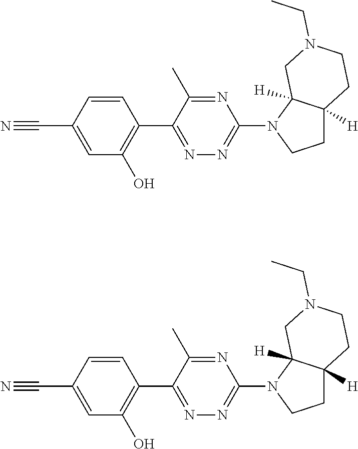 [SMILES: CCN1CC[C@H]2CCN(c3nnc(-c4ccc(C#N)cc4O)c(C)n3)[C@H]2C1 ; CCN1CC[C@@H]2CCN(c3nnc(-c4ccc(C#N)cc4O)c(C)n3)[C@@H]2C1]

Chiral separation by SFC (column chiral IK, eluent B: 40% methanol+0.2% diethylamine) of 4-[3-(6-ethyl-3,3a,4,5,7,7a-hexahydro-2H-pyrrolo[2,3-c]pyridin-1-yl)-5-methyl-1,2,4-triazin-6-yl]-3-hydroxy-benzonitrile (Example 2, step A) (62 mg, 0.16 mmol, 1.00 eq) to afford the two enantiomers example 2A (first eluting, Rt=2.41 minutes) (28 mg, 45% yield) as a brown foam; LCMS: m/z 365.2 [M+H]⁺, ESI pos and example 2B (second eluting, Rt=3.36 minutes) (28 mg, 45% yield) as a brown foam; LCMS: m/z 365.2 [M+H]⁺, ESI pos.

### Example 3A and 3B

### 5-[3-[(3aS,7aR)-6-Methyl-3,3,4,5,7,7a-hexahydro-2H-pyrrolo[2,3-c]pyridin-1-yl]-5-methyl-1,2,4-triazin-6-yl]-2,3-dihydrobenzofuran-4-ol and 5-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol

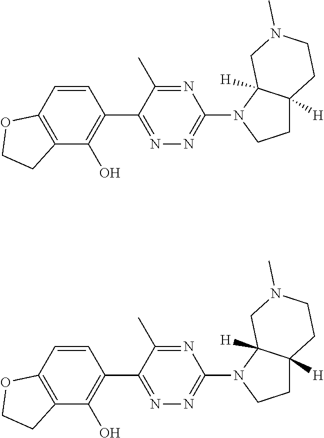 [SMILES: Cc1nc(N2CC[C@@H]3CCN(C)C[C@@H]32)nnc1-c1ccc2c(c1O)CCO2 ; Cc1nc(N2CC[C@H]3CCN(C)C[C@H]32)nnc1-c1ccc2c(c1O)CCO2]

### Step A: 1-(6-Chloro-5-methyl-1,2,4-triazin-3-yl)-6-methyl-3,3a,4,5,7,7a-hexahydro-2H-pyrrolo[2,3-c]pyridine

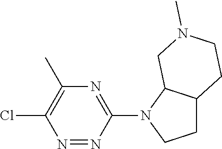 [SMILES: Cc1nc(N2CCC3CCN(C)CC32)nnc1Cl]

To a suspension of 1-(6-chloro-5-methyl-1,2,4-triazin-3-yl)-2,3,3a,4,5,6,7,7a-octahydropyrrolo[2,3-c]pyridine hydrochloride (Example 1, step B) (578 mg, 1.29 mmol, 1.00 eq, 65% purity) in 1,2-dichloroethane (12 mL) was added triethylamine (203 mg, 0.280 mL, 2.01 mmol, 1.55 eq). Let stir at room temperature for 5 minutes. Formaldehyde, 37% aqueous solution (218 mg, 0.20 mL, 2.69 mmol, 2.07 eq) was added followed by portion-wise addition of sodium triacetoxyborohydride (1.10 g, 5.18 mmol, 4.00 eq). The reaction mixture was stirred at room temperature for 1.5 hours. The reaction mixture was carefully quenched with saturated aq. NaHCO₃-solution and extracted four times with dichloromethane. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (335 mg, 92% yield) as a yellow oil. LCMS: m/z 268.1 [M+H]⁺, ESI pos.

### Step B: 5-[5-Methyl-3-(6-methyl-3,3a,4,5,7,7a-hexahydro-2H-pyrrolo[2,3-c]pyridin-1-yl)-1,2,4-triazin-6-yl]-2,3-dihydrobenzofuran-4-ol

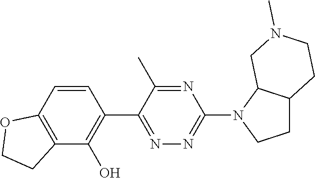 [SMILES: Cc1nc(N2CCC3CCN(C)CC32)nnc1-c1ccc2c(c1O)CCO2]

A mixture of 1-(6-chloro-5-methyl-1,2,4-triazin-3-yl)-6-methyl-3,3a,4,5,7,7a-hexahydro-2H-pyrrolo[2,3-c]pyridine (Example 3, step A) (160 mg, 0.57 mmol, 1.00 eq), 5-(4,4,5,5-tetramethyl-1,3,2-dioxaborolan-2-yl)-2,3-dihydrobenzofuran-4-ol (Example 1, step F) (251 mg, 0.91 mmol, 1.60 eq), potassium carbonate (360 mg, 2.60 mmol, 4.59 eq) and 1,1′-bis(diphenylphosphino) ferrocene-palladium(II) dichloride dichloromethane complex (70 mg, 0.09 mmol, 0.15 eq) in 1,4-dioxane (3.6 mL) and water (1.8 mL) was flushed with argon and stirred at 95° C. for 16 hours. The reaction mixture was cooled to room temperature and then extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 25 g, gradient 0% to 10% methanol in dichloromethane) to afford the title compound (173 mg, 75% yield, 90% purity) as a dark brown foam. LCMS: m/z 368.3 [M+H]⁺, ESI pos.

### Step C: 5-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol and 5-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol

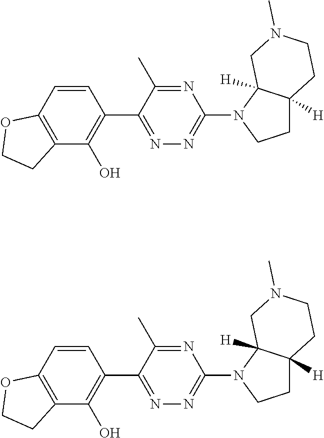 [SMILES: Cc1nc(N2CC[C@@H]3CCN(C)C[C@@H]32)nnc1-c1ccc2c(c1O)CCO2 ; Cc1nc(N2CC[C@H]3CCN(C)C[C@H]32)nnc1-c1ccc2c(c1O)CCO2]

Chiral separation by SFC (column chiral IK, eluent B: 33% methanol+0.2% diethylamine) of 5-[5-methyl-3-(6-methyl-3,3a,4,5,7,7a-hexahydro-2H-pyrrolo[2,3-c]pyridin-1-yl)-1,2,4-triazin-6-yl]-2,3-dihydrobenzofuran-4-ol (Example 3, step B) (173 mg, 0.42 mmol, 1.00 eq, 90% purity) to afford the two enantiomers example 3A (first eluting, Rt=2.37 minutes) (60 mg, 37% yield) as a yellow foam; LCMS: m/z 368.2 [M+H]⁺, ESI pos and example 3B (second eluting, Rt=2.55 minutes) (58 mg, 35% yield) as a yellow foam; LCMS: m/z 368.3 [M+H]⁺, ESI pos.

### Example 4A and 4B

### 4-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-3-hydroxy-benzonitrile and 4-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-3-hydroxy-benzonitrile

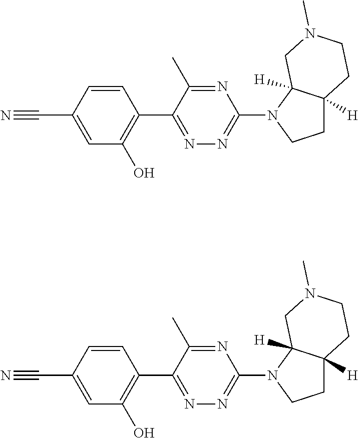 [SMILES: Cc1nc(N2CC[C@@H]3CCN(C)C[C@@H]32)nnc1-c1ccc(C#N)cc1O ; Cc1nc(N2CC[C@H]3CCN(C)C[C@H]32)nnc1-c1ccc(C#N)cc1O]

### Step A: 3-Hydroxy-4-[5-methyl-3-(6-methyl-3,3a,4,5,7,7a-hexahydro-2H-pyrrolo[2,3-c]pyridin-1-yl)-1,2,4-triazin-6-yl]benzonitrile

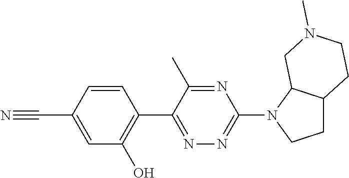 [SMILES: Cc1nc(N2CCC3CCN(C)CC32)nnc1-c1ccc(C#N)cc1O]

A mixture of 1-(6-chloro-5-methyl-1,2,4-triazin-3-yl)-6-methyl-3,3a,4,5,7,7a-hexahydro-2H-pyrrolo[2,3-c]pyridine (Example 3, step A) (160 mg, 0.57 mmol, 1.00 eq), (4-cyano-2-hydroxy-phenyl) boronic acid (commercially available, no CAS, 150 mg, 0.92 mmol, 1.62 eq), potassium carbonate (360 mg, 2.60 mmol, 4.59 eq) and 1,1′-bis(diphenylphosphino) ferrocene-palladium(II) dichloride dichloromethane complex (70 mg, 0.09 mmol, 0.15 eq) in 1,4-dioxane (3.6 mL) and water (1.8 mL) was flushed with argon and stirred at 95° C. for 16 hours. The reaction mixture was cooled to room temperature and then extracted with ethyl acetate and half-saturated NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 25 g, gradient 0% to 10% methanol in dichloromethane) to afford the title compound (112 mg, 53% yield) as a brown foam. LCMS: m/z 351.2 [M+H]⁺, ESI pos.

### Step B: 4-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-3-hydroxy-benzonitrile and 4-[3-[(3aR,7aS)-6-methyl-3.3a,4,5,7,7a-hexahydro-2H-pyrrolo[2,3-c]pyridin-1-yl]-5-methyl-1,2,4-triazin-6-yl]-3-hydroxy-benzonitrile

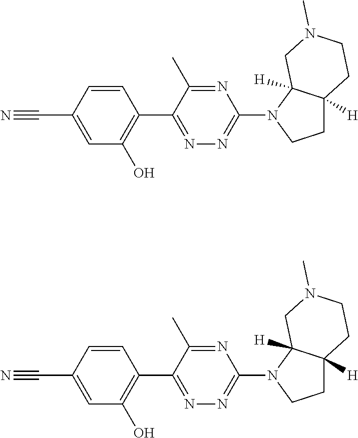 [SMILES: Cc1nc(N2CC[C@@H]3CCN(C)C[C@@H]32)nnc1-c1ccc(C#N)cc1O ; Cc1nc(N2CC[C@H]3CCN(C)C[C@H]32)nnc1-c1ccc(C#N)cc1O]

Chiral separation by SFC (column: achiral Torus2Pic, eluent B: 45% methanol+0.2% diethylamine) of 3-hydroxy-4-[5-methyl-3-(6-methyl-3,3a,4,5,7,7a-hexahydro-2H-pyrrolo[2,3-c]pyridin-1-yl)-1,2,4-triazin-6-yl]benzonitrile (Example 4, step A) (112 mg, 0.30 mmol, 1.00 eq) to afford the two enantiomers example 4A (first eluting, Rt=2.75 minutes) (52 mg, 46% yield) as a brown foam; LCMS: m/z 351.2 [M+H]⁺, ESI pos and example 4B (second eluting, Rt=3.20 minutes) (47 mg, 42% yield) as a brown foam; LCMS: m/z 351.3 [M+H]⁺, ESI pos.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula I 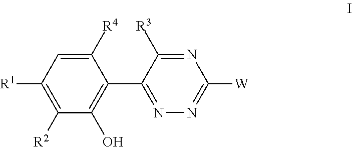
R¹ is H, halo, alkyl, haloalkyl, haloalkoxy or cyano;
R² is H;
or R¹ and R², and the atoms to which they are bonded, form a 4-6 membered cycloalkyl or a 4-6 membered heterocycle ring, wherein the cycloalkyl and the heterocycle ring are optionally substituted with one or two substituents independently selected from halo, alkyl or oxo;
R³ is selected from H, alkyl, cyano or haloalkyl, and R⁴ is selected from H, alkyl, halo, cyano or —CH₂-alkoxy, wherein R³ and R⁴ are not both H;
W is an 8-10 membered “N-linked” bicyclic ring system or an 8-10 membered “N-linked” spirocyclic ring system, wherein the N-Linked bicyclic ring system or N-Linked spirocyclic ring system comprise at least 2 N heteroatoms and can be optionally substituted by up to four substituents independently selected from hydroxyl, alkyl, halogen or cyano;
and pharmaceutically acceptable salts thereof.

2. A compound according to claim 1, R¹ is cyano or haloalkyl and R² is H, or R¹ and R², and the atoms to which they are bonded, form a 4-6 membered heterocycle ring comprising a single O heteroatom optionally substituted with one or two substituents independently selected from halo or alkyl.

3. A compound according to claim 1, wherein R¹ is cyano and R² is H, or R¹ and R², and the atoms to which they are bonded, form a 5 membered heterocycle ring comprising a single O heteroatom

4. A compound according to claim 1, wherein R³ is H or alkyl.

5. A compound according to claim 1, wherein R³ is alkyl.

6. A compound according to claim 1, wherein R⁴ is H.

7. A compound according to claim 1, wherein W is selected from ring systems A, B, or C 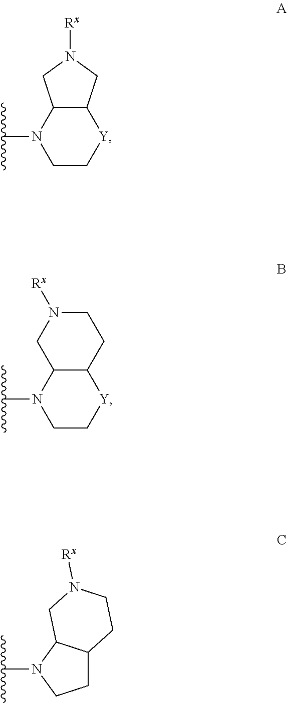
wherein
Y is CH₂, O, or NR^{y};
R^{y} is H or alkyl;
R^{x} is H or alkyl;
and the ring system A, B, or C can be further substituted with one cyano or up to two halo substituents.

8. A compound according to claim 7, wherein Y is CH₂ or O and R^{x} is H or alkyl.

9. A compound according to claim 1, wherein
R¹ is cyano and R² is H, or R¹ and R², and the atoms to which they are bonded, form a 5 membered heterocycle ring comprising a single O heteroatom; R³ is alkyl; R⁴ is HW is ring system C 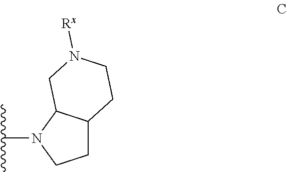
R^{x} is alkyl; and pharmaceutically acceptable salts thereof.

10. A compound according to any one of claims 1 to 9 claim 1, wherein the compound is selected from
5-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
5-[3-[(3aR,7aS)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aS,7aR)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
4-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile;
4-[3-[(3aR,7aS)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aS,7aR)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile;
5-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
5-[3-[(3aR,7aS)-6-Methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aS,7aR)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
4-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-3-hydroxy-benzonitrile;
4-[3-[(3aR,7aS)-6-Methyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aS,7aR)-6-methyl-3,3a,4,5,7,7a-hexahydro-2H-pyrrolo[2,3-c]pyridin-1-yl]-5-methyl-1,2,4-triazin-6-yl]-3-hydroxy-benzonitrile;
and pharmaceutically acceptable salts thereof.

11. A compound according to claim 1, wherein the compound is selected from
5-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
5-[3-[(3aR,7aS)-6-Ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aS,7aR)-6-ethyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
4-[3-[(3aS,7aR)-6-Ethyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aR,7aS)-6-ethyl-3,3a,4,5,7,7a-hexahydro-2H-pyrrolo[2,3-c]pyridin-1-yl]-5-methyl-1,2,4-triazin-6-yl]-3-hydroxy-benzonitrile;
5-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol or 5-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-2,3-dihydrobenzofuran-4-ol;
4-[3-[(3aS,7aR)-6-Methyl-3,3a,4,5,7,7a-hexahydro-2H-pyrrolo[2,3-c]pyridin-1-yl]-5-methyl-1,2,4-triazin-6-yl]-3-hydroxy-benzonitrile or 4-[3-[(3aR,7aS)-6-methyl-3,3a,4,5,7,7a-hexahydro-2H-pyrrolo[2,3-c]pyridin-1-yl]-5-methyl-1,2,4-triazin-6-yl]-3-hydroxy-benzonitrile;
and pharmaceutically acceptable salts thereof.

12. (canceled)

13. (canceled)

14. A pharmaceutical composition comprising a compound according to claim 1 and a therapeutically inert carrier.

15-21. (canceled)

22. A method of inhibiting NLRP3, which method comprises administering an effective amount of a compound as claimed in claim 1 to inhibit NLRP3.

23. A method for the treatment or prophylaxis of a disease, disorder or condition, which method comprises administering an effective amount of a compound according to claim 1 wherein the disease, disorder or condition is selected from Asthma or COPD.

24. A method for the treatment or prophylaxis of a disease, disorder or condition, which method comprises administering an effective amount of a compound according to claim 1, wherein the disease, disorder or condition is selected from Parkinson's Disease or Alzheimer's Disease.
